(19) United States
(12) Patent Application Publication
(10) Pub. No.: US 2025/0320666 A1
(43) Pub. Date: Oct. 16, 2025

(54) MOLDED FIBER CONTAINERS FOR A VARIETY OF BEVERAGES

(71) Applicant: Footprint International, LLC, Gilbert, AZ (US)

(72) Inventors: Victoria Kensy, Chandler, AZ (US); Yoke Chung, Gilbert, AZ (US); Kolbe Scheetz, Gilbert, AZ (US)

(21) Appl. No.: 19/176,039

(22) Filed: Apr. 10, 2025

Related U.S. Application Data
(60) Provisional application No. 63/633,027, filed on Apr. 11, 2024.

(51) Int. Cl. D21H 21/16; B65D 37/00; C07H 13/06; D21H 17/00; D21H 17/12

(52) U.S. Cl. CPC D21H 21/16; B65D 37/00; C07H 13/06; D21H 17/12; D21H 17/73

(57) ABSTRACT

Disclosed are methods, systems, and articles of manufacture for molded fiber beverage containers capable of containing a variety of fluids, including beverage cups for drinkable beverages, such as hot or cold beverages, carbonated beverages, and/or alcohol beverages. In some aspects, a method for producing a physical barrier to liquids for fibers usable in manufacturing of paper-based beverage containers includes dosing a fiber with a stearate or a saturated long chain fatty acid salt; dispersing undissolved stearate or undissolved saturated long chain fatty acid salt to allow for preparation of a homogenous mixture including the dosed fiber; and adding a divalent or trivalent metal salt to precipitate the stearate or the saturated long chain fatty acid salt from the homogenous mixture, wherein the precipitate is on the surface of the fiber and forms a physical barrier to liquids into the fiber.

### CROSS-REFERENCE TO RELATED APPLICATIONS

This application claims priority to and the benefit of U.S. Provisional Patent Application No. 63/633,027, filed Apr. 11, 2024, which is incorporated herein by reference in its entirety.

### TECHNICAL FIELD

The present technology relates, generally, to the manufacture of plastic-free, fiber-based products.

### BACKGROUND

Fiber-based packaging products are biodegradable, compostable and, unlike plastics, do not migrate into the ocean. Molded fiber is a packaging material typically made from a pulp of recycled paperboard and considered an environmentally sustainable packaging option. Molded pulp manufacturing has experienced increased popularity in recent years in a wide range of applications, including, for example, cups, bowls, straws, and the like.

### SUMMARY

Disclosed are methods, systems, and articles of manufacture for molded fiber beverage containers capable of containing a variety of fluids, including beverage cups for drinkable beverages, such as hot or cold beverages, carbonated beverages, and/or alcohol beverages. For example, embodiments of the disclosed molded fiber beverage cups are able to sustainably contain beverages (i.e., not undergo substantial degradation to the integrity of the beverage cup) at a wide range of temperatures (e.g., 0° C. (32° F.) to 72° C. (161° F.)) and for various types of beverages, such as those having an acidic or basic pH (e.g., pH 2.5 to pH 9.5) and/or containing alcohol (e.g., ethanol). The disclosed molded fiber beverage cups are able to sustainably contain alcoholic beverages in addition to non-alcoholic beverages for a vast range of drinking temperatures and pH content.

In some embodiments in accordance with the present technology, a method for producing a physical barrier to liquids for fibers usable in manufacturing of paper-based beverage containers includes dosing a fiber with a stearate or a saturated long chain fatty acid salt; dispersing undissolved stearate or undissolved saturated long chain fatty acid salt to allow for preparation of a homogenous mixture including the dosed fiber; and adding a divalent or trivalent metal salt to precipitate the stearate or the saturated long chain fatty acid salt from the homogenous mixture, wherein the precipitate is on the surface of the fiber and forms a physical barrier to liquids into the fiber.

The subject matter described in this patent document can be implemented in specific ways that provide one or more of the following features.

### BRIEF DESCRIPTION OF THE DRAWINGS

FIG. 1 shows a flow diagram of a method for producing a physical barrier to liquids for fibers usable in the production of molded fiber beverage containers, in accordance with the present technology.

FIG. 2 shows a flow diagram of a method for manufacturing molded fiber beverage containers capable of holding hot, cold, carbonated, and alcoholic beverages, in accordance with the present technology.

FIG. 3 shows a block diagram of an example embodiment of a data processing unit for an example embodiment of a system for manufacturing molded fiber beverage containers, in accordance with the present technology.

### DETAILED DESCRIPTION

Alkyl ketene dimer (AKD) is a common sizing agent used in the manufacturing of paper products, such as paper-based containers like cups, bowls, etc. Yet, due to the chemically reactivity and ability of AKD to modify cellulose, AKD has been deemed a single-use plastic and outlawed in some countries.

New techniques and non-plastic materials are needed for beverage containers that can hold hot, cold, carbonated and alcoholic beverages, for at least a substantial period of time (e.g., at least 3 hours).

Disclosed are methods, systems, and articles of manufacture for molded fiber beverage containers capable of containing a variety of fluids, including beverage cups for drinkable beverages, such as hot or cold beverages, carbonated beverages, and/or alcohol beverages. For example, embodiments of the disclosed molded fiber beverage cups are able to sustainably contain beverages (i.e., not undergo substantial degradation to the integrity of the beverage cup) at a wide range of temperatures (e.g., 0° C. (32° F.) and 72° C. (161° F.)) and for various types of beverages, such as those having an acidic or basic pH (e.g., pH 2.5 to pH 9.5) and/or containing alcohol (e.g., ethanol). The disclosed molded fiber beverage cups are able to sustainably contain alcoholic beverages in addition to non-alcoholic beverages for a vast range of drinking temperatures and pH content.

In some embodiments in accordance with the present technology, a method for producing a physical barrier to liquids for fibers usable in manufacturing of paper-based beverage containers includes dosing a fiber with a stearate or a saturated long chain fatty acid salt; dispersing undissolved stearate or undissolved saturated long chain fatty acid salt to allow for preparation of a homogenous mixture including the dosed fiber; and adding a divalent or trivalent metal salt to precipitate the stearate or the saturated long chain fatty acid salt from the homogenous mixture, wherein the precipitate is on the surface of the fiber and forms a physical barrier to liquids into the fiber.

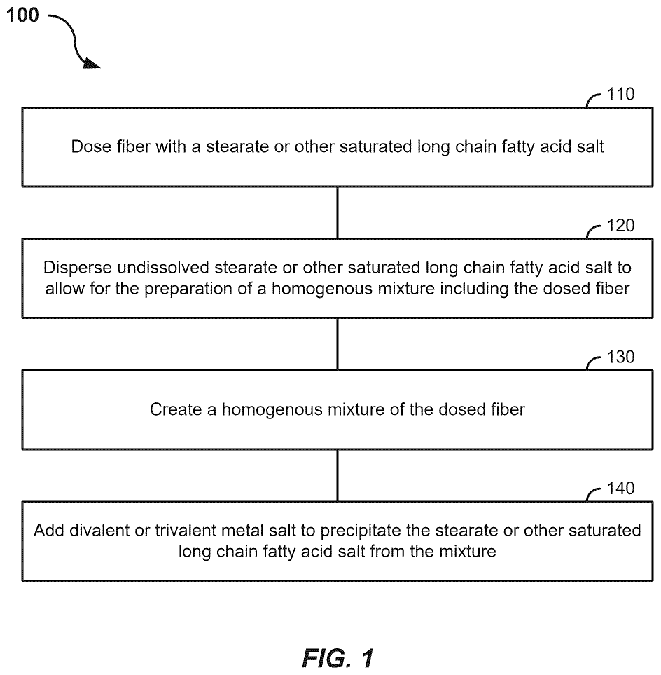

FIG. 1 shows a flow diagram of a method 100 for producing a physical barrier to liquids for fibers used in the production of paper-based beverage containers, in accordance with the present technology, which includes the following wet end chemistry and fiber formulation techniques.

The method 100 includes a process 110 to dose fiber with a stearate or other saturated long chain fatty acid salt (e.g., of sodium or potassium). The method 100 includes a process 120 to disperse undissolved stearate or other saturated long chain fatty acid salt to allow for the preparation of a homogenous mixture including the dosed fiber. The method 100 includes a process 130 to create a homogeneous mixture of the dosed fiber. The method 100 includes a process 140 to add a divalent or trivalent metal salt (e.g., divalent calcium salt) to precipitate the stearate or other saturated long chain fatty acid salt from the mixture, e.g., precipitate to the surface of the suspended fiber, thereby forming a physical barrier to liquids into the fiber.

In some embodiments of the process 110, the fiber is dosed with sodium stearate (C₁₈H₃₅NaO₂). In some implementations, for example, sodium stearate may be added to the fiber in a hot solution (e.g., a hot temperature in a range of ˜80° C. to ˜90° C.) at a particular concentration or range (e.g., concentration of 10% (w/w)). In some implementations, for example, sodium stearate may be added in an ambient dispersion (e.g., an ambient temperature in a range of ˜18° C. to ˜30° C.) at a particular concentration or range (e.g., concentration of 1% (w/w)). In implementations of the process 110 involving hot dosing or ambient dosing, for example, the dosing process can include a minimum agitation period of 30 seconds, e.g., with no maximum time limit after addition of the dispersion. In some implementations, for example, the fiber may be dosed with sodium stearate as a dry solid directly into a pulper. In some embodiments of the process 110, the fiber can be dosed with the stearate (or other saturated long chain fatty acid salt) by adding the stearate (or other saturated long chain fatty acid salt) in solid form (e.g., powder) to a pulper with the fibers before pulping.

In example implementations of the method 100, the wet end chemistry is dosed based on the total weight of the dry fiber. In some implementations of the process 110, the fiber is dosed with the stearate or other saturated long chain fatty acid salt at a concentration of 10% (w/w). For example, if a batch is prepared with 100 kg of dry fiber, 10 kg of sodium stearate would be added.

In some embodiments of the process 120, the undissolved stearate (or other saturated long chain fatty acid salt) can be dispersed during the addition of the stearate (or other saturated long chain fatty acid salt) to the fiber mixture when added via a gear metering pump. In some embodiments of the process 120, the undissolved stearate (or other saturated long chain fatty acid salt) can be dispersed via manual addition, e.g., such as a physically emptying a container of the undissolved stearate (or other saturated long chain fatty acid salt) into the fiber slurry tank. For example, sodium stearate is somewhat soluble, and thus some or all of the sodium stearate can dissolve within the fiber suspension. The dissolution of some/all of the sodium stearate and dispersion of any undissolved sodium stearate allows for the preparation of a homogenous mixture.

In the process 130, for example, the mixture is homogenized to provide an equal (or substantially equal) distribution of the stearate salt (or other saturated long chain fatty acid salt) throughout the mixture. This is achieved in some embodiments of the process 130 by mixing or agitating the mixture for a time period after the addition of the stearate or other saturated long chain fatty acid salt and before the addition of the di/tri-valent metal salt. In some implementations of the process 130, for example, the time period for reaching a homogenous state is proportional to the mixing efficiency and may vary based on the shape and rpms (rotations per minute) of the agitator apparatus. For example, for some agitator apparatuses, the time period of a mix time can range between 30 seconds to 10 minutes.

After generating a homogenous mixture, for example, the divalent calcium salt is added to precipitate the dissolved sodium stearate from within the fiber as calcium stearate on the surface of the suspended fiber. For example, the precipitation of the calcium stearate on the fiber provides a uniform distribution of chemistry on the fiber that has been shown to be important for consistent performance. For instance, the calcium stearate on the surface of the fiber provides a physical barrier to liquids. In some embodiments of the process 140, the divalent or trivalent metal salt is typically dissolved at ambient conditions, e.g., at around 12.5% solids content. It can be prepared at higher temperatures and higher solids content as well, e.g., up to 100° C. and 50% solid content. In some implementations of the process 140, the divalent or trivalent metal salt solution can be added via a gear metering pump. In some implementations of the process 140, the divalent or trivalent metal salt solution can be manually added manually, e.g., via a container of the salt solution. In some implementations of the process 140, the divalent or trivalent metal salt solution can be dispersed as a solid into the fiber slurry. In various implementations of the process 140, a full amount of the salt is dosed all at once, e.g., up to the 2.74% dry weight based on total weight of the dry fiber.

In some embodiments of the method 100, sodium stearate can be substituted with potassium stearate or other saturated long chain fatty acid salts (e.g., C16 through C25) of sodium or potassium. Soluble divalent or trivalent salts of magnesium, aluminum, zinc, calcium, or iron may be used to drive the precipitation of a carboxylate salt on the surface of the fiber. Sodium stearate has a greater solubility than several other stearate salts, which is beneficial for achieving a homogeneous distribution. In some embodiments of the process 140, calcium chloride is an example divalent metal salt that can be added. In general, the carboxylate salt will have a formula of (R—CO₂⁻)ₙM, where R is a saturated hydrocarbon chain of C15 through C24, where n is the number of carboxylate anions associated with a metal in the solid state, and where M is a metal cation with a charge of 2+ or 3+.

For example, a divalent metal with stearate can include: (C₁₇H₃₅CO₂)₂Ca. For example, a trivalent metal with stearate can include: (C₁₇H₃₅CO₂)₃Al.

In some embodiments of the method 100, for example, the method 100 optionally may include a process (not shown in FIG. 1) to condition the fiber by incorporating one or more additives to the fiber. For example, to enhance structural rigidity of the fiber, a strength additive may be incorporated into, placed on the surface of, or otherwise incorporated into the fiber. Strength additives can include, but are not limited to, liquid starches available commercially as Topcat® L98 cationic additive, manufactured by Ingredion Inc. of Westchester, IL, and/or Hercobond®, manufactured by Solenis International of Wilmington, DE, in a range of 0.1% to 5%, e.g., preferably 0.5% to 2.5% for some implementations, and for some implementations be about 2.0%±1%. Alternatively or in addition, the liquid starch may be combined with low charge liquid cationic starches such as those available as Penbond® cationic additive, manufactured by Ingredion Inc., and PAF 9137 BR cationic additive to achieve the range of 0.1% to 5%, e.g., preferably 0.5% to 2.0% for some implementations. Also, for example, additives to increase the dry strength may be conditioned with other starches. Examples include polyamide-epichlorohydrin (PAE) resins, a commercially available wet-strength additive such as Kymene™ 920A and/or wet-strength resin Kymene™ 1500, manufactured by Solenis International, or other wet strength additives to achieve the range of 0.1% to 5%, e.g., preferably 0.5% to 2.0% for some implementations. Penbond® is a medium charge, cationic polymer; Topcat® is a high-charge cationic additive; and Hercobond® includes amphotheric, anionic and cationic polyacrylamides, cationic glyoxylated resins, modified polyamines, and cellulase enzymes.

Table 1 shows example constituents for a molded fiber product formulation in accordance with some embodiments of the method 100. Functional range is a range of chemical addition (by percentage of total weight of dry fiber) at which a molded fiber product still demonstrates the specified capabilities for beverage containment.

TABLE 1

| | Recipe % based | | |
|---|---|---|---|
| | on total | Functional | |
| Component | weight of dry fiber | Range (%) | Function |
| Sodium | 10.00% | 1.5%-12% or | Supports barrier |
| Stearate | | potentially higher | formation. |
| Calcium | 2.74% | 0.69%-12% or | Supports barrier |
| chloride | | potentially higher | formation. |
| Dry-strength | 0.50% | 0 %-3.0% | Processing aid |
| additive (e.g., | | | |
| Hercobond ® | | | |
| 6950) | | | |

In some implementations of the method 100, substitution of an unbleached hard or soft wood fiber into the molded fiber beverage container formulation may be performed, as it is not expected to impact product functionality.

Table 2 shows an example molded fiber product formulation for some embodiments of the method 100.

TABLE 2
Fiber

| | Recipe % based | Functional |
|---|---|---|
| Component | on dry weight | Range (%) |
| Bleached softwood | 50% | 0-100% |
| Bleached hardwood | 50% | 0-100% |

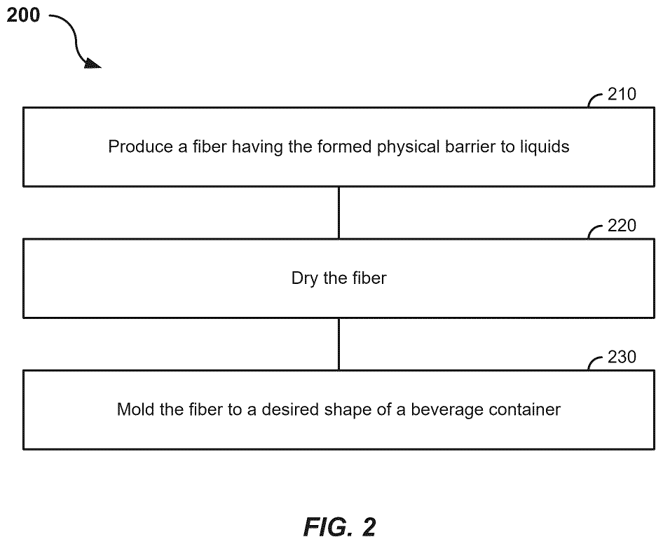

FIG. 2 shows a flow diagram of a method 200 for manufacturing molded fiber beverage containers capable of holding hot, cold, carbonated, and alcoholic beverages, in accordance with the present technology. The method 200 includes a process 210 to produce a fiber having the formed physical barrier to liquids. Implementations of the process 210 includes implementation of an example embodiment of the method 100. The method 200 includes a process 220 to dry the fiber having the formed physical barrier to liquids. The method 200 includes a process to mold the fiber to the desired shape of a beverage container for a final product. In some embodiments of the method 200, the process 220 precedes the process 230; whereas in some embodiments of the method 200, the process 230 precedes the process 220; or, whereas in some embodiments of the method 200, the processes 220 and 230 are implemented concurrently or at least partially concurrently. Implementations of the method 200 can produce a molded fiber beverage container (e.g., cup, bowl, or other container type) that is able to sustainably contain a beverage (i.e., not undergo substantial degradation to the integrity of the beverage cup) at a wide range of temperatures (e.g., 0° C. (32° F.) to 72° C. (161° F.)) and for various types of beverages, such as those having an acidic or basic pH (e.g., pH 2.5 to pH 9.5) and/or containing alcohol (e.g., ethanol). The produced molded fiber beverage container is able to sustainably contain alcoholic beverages in addition to non-alcoholic beverages for a vast range of drinking temperatures and pH content.

For example, in some implementations of the method 200, the process 220 and 230 are implemented together in a dry-molding method, e.g., where the processes 220 and 230 can be implemented using a dry-molding system that dry-molds the fiber (having the liquid barrier) under a heat and pressure regime to form a finished molded fiber container product. An exemplary dry molding method (for the processes 220 and 230) can include a sequence of three processes: a perforation (or pre-slitting) process, a pressing process, and a cutting process. The method 200 may optionally include a process 240 to transport a finished molded fiber container product from a dry-molding system for product handling (e.g., storage and/or distribution).

In such embodiments, for example, the perforation process may include cutting, stamping, and/or slitting the fiber in a predetermined arrangement and to a predetermined depth to facilitate deformation of the fiber during subsequent steps. For example, cutting members (e.g., blades) can be arranged in patterns and/or moved to perforate the fiber to create a pattern of cut lines (e.g., to cross-cut the fiber). The perforation process can provide a form of stress-relief that prevents or reduces the likelihood tearing and folding of fiber during its shape forming, as well as enable deeper draw products and reduced trim waste. This allows multiple individual mold tools to be located adjacent to each other such that multiple different molded pieces can be formed at the same time during a single forming operation (referred to as “cavitation”).

After the perforation process, the perforated fiber a single-pressing process or multi-pressing process. For example, for a single-pressing process, either hot or cold forming pressing can be performed, in which the perforated fiber is pressed between upper and lower presses of a pressing apparatus, thereby producing a preliminary product structure from the fiber. The force, speed, and surface features of the upper and lower presses of the pressing apparatus can vary depending upon what the desired properties, structure, and nature are to be for the final beverage container product. Also, for example, for a multi-pressing process, a first pressing process can involve pressing the perforated fiber to an optimized percentage (e.g., 5%-90%) prior to final pressing process that can pre-shape the first-pressed fiber structure to prepare for the preliminary product structure before the final cutting process to produce the final product.

In some implementations of the pressing process, for example, forming conditions may include low to high pressure (e.g., 20-4000 psi) and/or low to high temperature (from standard room temperature to 500° F., for example), depending upon the application. In some implementations of the pressing process, for example, hot-pressing/forming can be performed using a prescribed moisture content range from about 0-25%. The press temperature may range from 210° F. to 500° F. Pressure for dry-forming fiber may range from 22-6,000 psi, e.g., depending on product geometry and other factors. The dwell time during which the forming pressure is applied for either or both of a single-pressing or a multi-pressing process may be of a duration between 0.1-3.0 seconds.

After the pressing process, the cutting process is performed to cut the pressed fiber into a final product. In some implementations, for example, the cutting process includes a die cutting process.

Drying and pressing of the beverage container in the manufacturing process have been shown to be important to its performance. Beverage containers (e.g., cups, bowls, etc.) that are not well pressed or fully dried do sufficiently hold liquid beverages.

Example embodiments of a system for manufacturing molded fiber beverage containers can include a plurality of components controllable by a controller device of the system, e.g., embodied by a data processing unit. Examples of the plurality of components for the system include, but are not limited to, one or more fluid dispensing units, such as an apparatus comprising a nozzle and valve and/or pump, e.g., to regulate the flow of fluid out of the nozzle; one or more heating units, such as an induction heater device or a radiation heater device, e.g., to bring materials, like treated fiber, to a desired temperature for curing, drying, facilitating chemical reactions or diffusion, etc., depending upon the application; UV lamps, e.g., to sterilize, cure, etc. materials; sensors to detect a property or condition of a material or portion of the system; and/or fan, conveyer belt, or other mechanism(s) to affect an aspect of the manufacturing process, e.g., cause laminar or turbulent flow within a chamber of the system.

One example embodiment of a system for manufacturing a molded fiber beverage container, in accordance with the present technology, includes a fiber production apparatus, a cutting apparatus, and a paper product forming apparatus. The fiber production apparatus includes one or more fluid dispensing units and one or more heating units, configured to produce a fiber sheet (e.g., a layer or other piece of fiber) having a physical barrier to liquids by: dosing a fiber with a stearate or a saturated long chain fatty acid salt, dispersing undissolved stearate or undissolved saturated long chain fatty acid salt to allow for preparation of a homogenous mixture including the dosed fiber, and adding a divalent or trivalent metal salt to precipitate the stearate or the saturated long chain fatty acid salt from the homogenous mixture, where the precipitate is on a surface of the fiber and forms a physical barrier to liquids into the fiber, wherein the fiber is formed as the fiber sheet. The cutting apparatus includes one or more blades to cross-cut the fiber sheet into fiber sections. The paper product forming apparatus includes at least one slitting tool, at least one pressing tool, and at least one cutting tool, configured to mold one or more portions of the fiber sections under one or more of heat, wet chemicals, or pressure to produce a plurality of finished paper-based beverage container products. In some implementations of the system, for example, the plurality of finished paper-based beverage container products is able to perform at least one of the following: (i) sustainably contain a beverage for at least three hours that is at a temperature in a range of 0° C. to 72° C., (ii) sustainably contain a beverage for at least three hours that has an acidic pH or a basic pH in a range of pH 2.5 to pH 9.5, or (iii) sustainably contain a beverage for at least three hours that contains an alcohol.

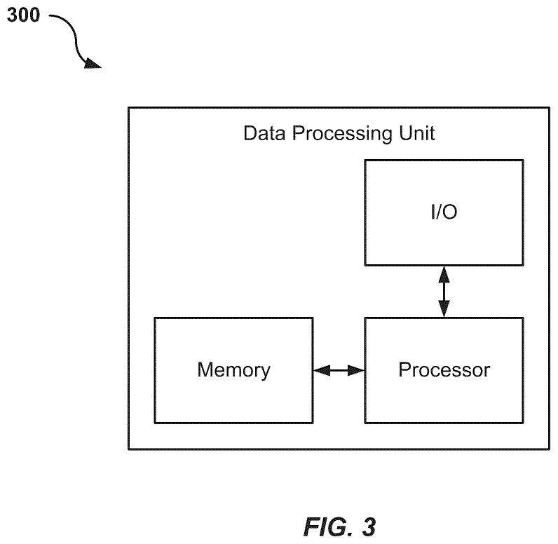

FIG. 3 shows a block diagram of an example embodiment of a data processing unit 300 for some example embodiments of a system for manufacturing molded fiber beverage containers (not shown). The system may include one or multiple data processing units 300 associated with a section of the system. The data processing unit 300 of the system includes at least one processor to process data, at least one memory in communication with the processor(s) to store data, and/or an input/output unit (I/O) to interface the processor(s) and/or memory(ies) to other sections, modules, units, or devices of the system or external devices.

For example, the processor of the data processing unit can include a central processing unit (CPU), a graphics processing unit (GPU), and/or a microcontroller unit (MCU). For example, the memory can include and store processor-executable code, which when executed by the processor, configures the data processing unit to perform various operations, e.g., such as receiving information, commands, and/or data, processing information and data, and transmitting or providing information/data to another device. In some implementations, the data processing unit can transmit raw or processed data to a computer system or communication network accessible via the Internet (referred to as ‘the cloud’) that includes one or more remote computational processing devices (e.g., servers in the cloud). To support various functions of the data processing unit, the memory can store information and data, such as instructions, software, values, images, and other data processed or referenced by the processor. For example, various types of Random Access Memory (RAM) devices, Read Only Memory (ROM) devices, Flash Memory devices, and other suitable storage media can be used to implement storage functions of the memory unit. The I/O of the data processing unit can interface the data processing unit with the wireless communications unit to utilize various types of wired or wireless interfaces compatible with typical data communication standards, for example, which can be used in communications of the data processing unit with other devices, such as a wired or wireless communication device (e.g., tablet, laptop, smartphone or other computer or computing device) in communication with the data processing unit of the system. For example, the data processing unit can be wireless communication, via a wireless transmitter/receiver (Tx/Rx) unit, e.g., including, but not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G/6G cellular communication methods, NFC (Near Field Communication), and/or parallel interfaces, or other. The I/O of the data processing unit can also interface with other external interfaces, sources of data storage, and/or visual or audio display devices, etc. to retrieve and transfer data and information that can be processed by the processor, stored in the memory unit, or exhibited on an output unit of the system or an external device. For example, a display unit of the system can be configured to be in data communication with the data processing unit, e.g., via the I/O, to provide a visual display, an audio display, and/or other sensory display that produces the user interface of the software application of the disclosed technology for health management. In some examples, the display unit can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

In many cases, the techniques relating to additives, paper composition, and the like for wet molding may apply to dry molding in accordance with the present technology. In that regard, the systems and methods described above may incorporate by reference the disclosures of the following patent documents in their entirety and for all purposes: U.S. Pat. Pub. No. 2020/0206984, “Methods, Apparatus, and Chemical Compositions for Selectively Coating Fiber-Based Food Containers,” U.S. Ser. No. 10/428,467, “Methods and Apparatus for Manufacturing Fiber-Based Meat Products,” U.S. Pat. No. 9,988,199, “Methods and Apparatus for Manufacturing Fiber-Based Microwavable Food Containers,” U.S. Ser. No. 10/036,126, “Methods for Manufacturing Fiber-Based Beverage Lids,” U.S. Ser. No. 10/124,926, “Methods and Apparatus for Manufacturing Fiber-Based, Foldable Packaging Assemblies,” U.S. Pat. No. 9,856,608, “Methods for Manufacturing Fiber-Based Product Containers,” U.S. Ser. No. 10/087,584, “Methods and Apparatus for Manufacturing Fiber-Based Meat Containers,” U.S. Pat. No. 9,869,062, “Method for Manufacturing Microwavable Food Containers,” U.S. Ser. No. 10/377,547, “Method and Apparatus for In-line Die Cutting of Vacuum Formed Molded Pulp Container,” U.S. Ser. No. 10/240,286, “Die Press Assembly for Drying and Cutting Molded Fiber Parts,” U.S. Ser. No. 10/683,611, “Method for Simultaneously Pressing and Cutting a Molded Fiber Part,” and U.S. patent application Ser. No. 18/980,753, “Systems and Methods for Web-Fed Dry Forming of Fiber-Based Products.”

### EXAMPLES

In some embodiments in accordance with the present technology (example A1), a method for producing a physical barrier to liquids for fibers usable in manufacturing of paper-based beverage containers includes dosing a fiber with a stearate or a saturated long chain fatty acid salt; dispersing undissolved stearate or undissolved saturated long chain fatty acid salt to allow for preparation of a homogenous mixture including the dosed fiber; and adding a divalent or trivalent metal salt to precipitate the stearate or the saturated long chain fatty acid salt from the homogenous mixture, wherein the precipitate is on the surface of the fiber and forms a physical barrier to liquids into the fiber.

Example A2 includes the method of example A1 or any of examples A1-A16, wherein the stearate includes sodium stearate.

Example A3 includes the method of example A2 or any of examples A1-A16, wherein the dosing of the fiber with the sodium stearate includes adding the sodium stearate to the fiber in a hot solution at a temperature in a range of 80° C. to 90° C. at a concentration of 10% (w/w).

Example A4 includes the method of example A2 or any of examples A1-A16, wherein the dosing of the fiber with the sodium stearate includes adding the sodium stearate to the fiber in an ambient dispersion at a temperature in a range of 18° C. to 30° C. at a concentration of 1% (w/w).

Example A5 includes the method of example A2 or any of examples A1-A16, wherein the dosing of the fiber with the sodium stearate includes dosing as a dry solid directly into a pulper.

Example A6 includes the method of example A1 or any of examples A1-A16, wherein the divalent or trivalent metal salt includes a divalent calcium salt.

Example A7 includes the method of example A6 or any of examples A1-A16, wherein the divalent calcium salt precipitates as calcium stearate on the surface of the fiber to provide the physical barrier to liquids.

Example A8 includes the method of example A1 or any of examples A1-A16, wherein the divalent or trivalent metal salt includes one or more of calcium, magnesium, aluminum, zinc, or iron, and wherein the divalent or trivalent metal salt drives precipitation of a carboxylate salt on the surface of the fiber.

Example A9 includes the method of example A8 or any of examples A1-A16, wherein the carboxylate salt includes a formula including (R—CO₂⁻)ₙM, where R is a saturated hydrocarbon chain of C15 thru C24, n is a number of carboxylate anions associated with a metal in a solid state, and M is a metal cation with a charge of 2+ or 3+.

Example A10 includes the method of example A1 or any of examples A1-A16, wherein the method further comprises creating a homogeneous mixture of the dosed fiber.

Example A11 includes the method of example A1 or any of examples A1-A16, wherein the saturated long chain fatty acid salt includes at least one of sodium or potassium and includes a chain length of C16 to C25.

Example A12 includes the method of example A1 or any of examples A1-A16, wherein the method includes a method for manufacturing a molded fiber beverage container that comprises producing a fiber having a physical barrier to liquids in accordance with any of examples A1-A11; and drying and molding the fiber to produce a beverage container.

Example A13 includes the method of example A12 or any of examples A12-A16, wherein the produced beverage container includes a cup or a bowl.

Example A14 includes the method of example A12 or any of examples A12-A16, wherein the produced beverage container is able to sustainably contain a beverage for at least three hours that is at a temperature in a range of 0° C. to 72° C.

Example A15 includes the method of example A12 or any of examples A12-A16, wherein the produced beverage container is able to sustainably contain a beverage for at least three hours that has an acidic pH or a basic pH in a range of pH 2.5 to pH 9.5.

Example A16 includes the method of example A12 or any of examples A12-A15, wherein the produced beverage container is able to sustainably contain a beverage for at least three hours that contains an alcohol.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media, and memory devices, including by way of example semiconductor memory devices, e.g., EPROM, EEPROM, and flash memory devices. The processor and the memory can be supplemented by, or incorporated in, special purpose logic circuitry.

It will be appreciated that specific embodiments have been described herein for purposes of illustration but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein. The above detailed description of embodiments of the technology is not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method for producing a physical barrier to liquids for fibers for manufacturing of paper-based beverage containers, comprising:
dosing a fiber with a stearate or a saturated long chain fatty acid salt;
dispersing undissolved stearate or undissolved saturated long chain fatty acid salt to allow for preparation of a homogenous mixture including the dosed fiber; and
adding a divalent or trivalent metal salt to precipitate the stearate or the saturated long chain fatty acid salt from the homogenous mixture, wherein the precipitate is on a surface of the fiber and forms a physical barrier to liquids into the fiber.

2. The method of claim 1, wherein the stearate includes sodium stearate or potassium stearate.

3. The method of claim 2, wherein the dosing of the fiber with the sodium stearate includes adding the sodium stearate to the fiber in a hot solution at a temperature in a range of 80° C. to 90° C. at a concentration of 10% (w/w).

4. The method of claim 2, wherein the dosing of the fiber with the sodium stearate includes adding the sodium stearate to the fiber in an ambient dispersion at a temperature in a range of 18° C. to 30° C. at a concentration of 1% (w/w).

5. The method of claim 2, wherein the dosing of the fiber with the sodium stearate includes dosing as a dry solid directly into a pulper.

6. The method of claim 1, wherein the divalent or trivalent metal salt includes a divalent calcium salt.

7. The method of claim 6, wherein the divalent calcium salt precipitates as calcium stearate on the surface of the fiber to provide the physical barrier to liquids.

8. The method of claim 1, wherein the divalent or trivalent metal salt includes one or more of calcium, magnesium, aluminum, zinc, or iron, and wherein the divalent or trivalent metal salt drives precipitation of a carboxylate salt on the surface of the fiber.

9. The method of claim 8, wherein the carboxylate salt includes a formula including (R—CO₂⁻)ₙM, where R is a saturated hydrocarbon chain of C15 thru C24, n is a number of carboxylate anions associated with a metal in a solid state, and M is a metal cation with a charge of 2+ or 3+.

10. The method of claim 9, wherein the divalent metal salt includes (C₁₇H₃₅CO₂)₂Ca.

11. The method of claim 9, wherein the trivalent metal salt includes (C₁₇H₃₅CO₂)₃Al.

12. The method of claim 1, further comprising:
creating a homogeneous mixture of the dosed fiber.

13. The method of claim 1, wherein the saturated long chain fatty acid salt includes at least one of sodium or potassium and includes a chain length of C16 to C25.

14. A method for manufacturing a molded fiber beverage container, comprising:
producing a fiber having a physical barrier to liquids in accordance with the method of claim 1; and
drying and molding the fiber to produce a beverage container.

15. The method of claim 14, wherein the produced beverage container includes a cup or a bowl.

16. The method of claim 14, wherein the produced beverage container is able to sustainably contain a beverage for at least three hours that is at a temperature in a range of 0° C. to 72° C.

17. The method of claim 14, wherein the produced beverage container is able to sustainably contain a beverage for at least three hours that has an acidic pH or a basic pH in a range of pH 2.5 to pH 9.5.

18. The method of claim 14, wherein the produced beverage container is able to sustainably contain a beverage for at least three hours that contains an alcohol.

19. A system for manufacturing a molded fiber beverage container, comprising:
a fiber production apparatus, comprising one or more fluid dispensing units and one or more heating units, configured to produce a fiber sheet having a physical barrier to liquids by:
dosing a fiber with a stearate or a saturated long chain fatty acid salt,
dispersing undissolved stearate or undissolved saturated long chain fatty acid salt to allow for preparation of a homogenous mixture including the dosed fiber, and
adding a divalent or trivalent metal salt to precipitate the stearate or the saturated long chain fatty acid salt from the homogenous mixture, wherein the precipitate is on a surface of the fiber and forms a physical barrier to liquids into the fiber, wherein the fiber is formed as the fiber sheet;
a cutting apparatus, comprising one or more blades to cross-cut the fiber sheet into fiber sections; and
a paper product forming apparatus, comprising at least one slitting tool, at least one pressing tool, and at least one cutting tool, configured to mold one or more portions of the fiber sections under one or more of heat, wet chemicals, or pressure to produce a plurality of finished paper-based beverage container products.

20. The system of claim 19, wherein the plurality of finished paper-based beverage container products is able to perform at least one of the following: (i) sustainably contain a beverage for at least three hours that is at a temperature in a range of 0° C. to 72° C., (ii) sustainably contain a beverage for at least three hours that has an acidic pH or a basic pH in a range of pH 2.5 to pH 9.5, or (iii) sustainably contain a beverage for at least three hours that contains an alcohol.
